(19) United States
(12) Patent Application Publication
(10) Pub. No.: US 2020/0139004 A1
(43) Pub. Date: May 7, 2020

(54) METHOD FOR COATING CALCIUM SILICATE FOR PREPARING BONE GRAFTING MATERIAL HAVING IMPROVED BIOCOMPATIBILITY

(71) Applicants: Genoss Co., Ltd., Gyeonggi-do (KR); Gentren, Chungcheongnam-do (KR)

(72) Inventors: Sung-Min Chung, Gyeonggi-do (KR); Byung-Ho Yoon, Gyeonggi-do (KR); Yoon Ho Cho, Gyeonggi-do (KR); Seongok Na, Chungcheongbuk-do (KR)

(21) Appl. No.: 16/533,001

(22) Filed: Aug. 6, 2019

(30) Foreign Application Priority Data
Oct. 12, 2018 (KR) 10-2018-0122067

(51) Int. Cl. A61L 27/10; A61L 27/12; A61L 27/02

(52) U.S. Cl. CPC A61L 27/10; A61L 27/12; A61L 27/025; C03B 19/06; A61L 2430/02; A61L 2300/102; A61L 2400/06

(57) ABSTRACT

The present invention relates to a method for preparing a glass-ceramic composite, in which a bioactive material is uniformly coated on the surface of a ceramic molded body; a glass-ceramic composite, in which a bioactive material is uniformly coated on the surface of a ceramic molded body; a bone grafting material comprising the glass-ceramic composite; and a bone grafting kit comprising the glass-ceramic composite.

### CROSS REFERENCE TO RELATED APPLICATIONS

This application claims priority to Korean Patent Application No. 10-2018-0122067, filed on Oct. 12, 2018, the entire content of which is hereby incorporated by reference herein.

### TECHNICAL FIELD

The present invention relates to a method for preparing a glass-ceramic composite, in which a bioactive material is uniformly coated on the surface of a ceramic molded body; a glass-ceramic composite, in which a bioactive material is uniformly coated on the surface of a ceramic molded body; a bone grafting material comprising the glass-ceramic composite; and a bone grafting kit comprising the glass-ceramic composite.

### BACKGROUND ART

If there is insufficient residual bone volume associated with infectious disease, trauma, or implant placement, reconstruction using a bone graft is performed to restore defective bone tissues. For this purpose, it is accompanied by osteoinduction regeneration, or among the various types of bone grafting materials used alone, autogenous bone is considered to be an ideal bone grafting material having all of the properties of bone formation, osteoinduction, and bone conduction, but additional surgical sites are needed for taking. Accordingly, it has the disadvantage of increasing the likelihood of patient inconvenience and complications. Because of these problems, many studies have been made on various types of bone grafting materials to replace autogenous bones. In particular, studies on synthetic bone grafting materials have been extensively conducted, and it has been reported that synthetic bone grafting materials account for a significant portion of the graft materials used in bone reconstruction surgery worldwide. Among the ceramic materials most commonly used as synthetic bone substitutes, calcium phosphate-based materials are similar in terms of components and structures to bone tissues and have biocompatibility, and thus have been extensively studied as bone grafting materials. Among the calcium phosphate-based ceramics, hydroxyapatite (HA) and tricalcium phosphate (TCP), or biphasic calcium phosphate (BCP), which is a mixture of these two phases, have excellentkll and tissue responses by immobilization of various components, such as peptides, enzymes, and extracellular matrix (ECM). Accordingly, the present inventors have made efforts to develop a novel coated bone grafting material capable of promoting osteogenesis, synostosis, biocompatibility, and natural healing process of bone tissues after bone graft by coating a bioactive material, thereby completing the present invention.

### PRIOR ART DOCUMENT

### Patent Document

Korean Patent No. 10-1395533

### DISCLOSURE

### Technical Problem

It is one object of the present invention to provide a method for preparing a glass-ceramic composite, including:

Step 1 of preparing a slurry containing a ceramic powder;

Step 2 of forming a pore in a molded body by casting the slurry;

Step 3 of removing moisture by freeze-drying the molded body having the pore;

Step 4 of subjecting the molded body from which moisture is removed to primary sintering at 900° C. to 1100° C.;

Step 5 of coating the primary sintered molded body with a bioactive material; and

Step 6 of subjecting the coated molded body to secondary sintering at 1100° C. to 1300° C.

It is another object of the present invention to provide a glass-ceramic composite having a structure in which a bioactive material is coated on the surface of the ceramic molded body, wherein the bioactive material has an average particle size of 10 μm or less and is embedded in the surface of a ceramic molded body.

It is still another object of the present invention to provide a bone grafting material including the glass-ceramic composite having a structure in which a bioactive material is coated on the surface of the ceramic molded body, wherein the bioactive material has an average particle size of 10 μm or less and is embedded in the surface of a ceramic molded body.

It is still further another object of the present invention to provide a bone grafting kit including the glass-ceramic composite having a structure in which a bioactive material is coated on the surface of the ceramic molded body, wherein the bioactive material has an average particle size of 10 μm or less and is embedded in the surface of a ceramic molded body.

### Technical Solution

The present inventors have found that when a bioactive material is added to ceramics as a bone grafting material, a bone grafting material having excellent biocompatibility, bone forming-ability, antimicrobial activity, and increased wettability can be prepared. Accordingly, the present invention has been devised to provide a glass-ceramic composite in which a bioactive material is coated uniformly on a ceramic surface, which is characterized in that the bonding force between the bioactive material and the ceramic surface is improved, thereby reducing leaking of coating layer components and/or detachment of the coating layer itself.

The present invention will be described in detail below. Meanwhile, each description and embodiment disclosed herein can be applied to other descriptions and embodiments, respectively. That is, all combinations of various elements disclosed herein fall within the scope of the present invention. Further, the scope of the present invention is not limited by the specific description described below.

In first aspect, the present invention provides a method for preparing a glass-ceramic composite, including:

Step 1 of preparing a slurry containing a ceramic powder;

Step 2 of forming a pore in a molded body by casting the slurry;

Step 3 of removing moisture by freeze-drying the molded body having the pore;

Step 4 of subjecting the molded body from which moisture is removed to primary sintering at 900° C. to 1100° C.;

Step 5 of coating the primary sintered molded body with a bioactive material; and Step 6 of subjecting the coated molded body to secondary sintering at 1100° C. to 1300° C.

Hereinafter, in order to clarify and clearly distinguish the sintering steps of the present invention, the ‘primary sintering’ is used interchangeably with ‘pre-sintering’, and the ‘secondary sintering’ is used interchangeably with ‘complete-sintering’.

In the preparation method of the present invention, the ceramic powder in Step 1 may include at least one selected from the group consisting of hydroxyapatite (HA), biphasic calcium phosphate (BCP), tricalcium phosphate, and zirconia, but is not limited thereto.

In the preparation method of the present invention, Step 2 of forming a pore in a molded body by casting the slurry may be performed by a vacuum-assisted foaming technique, but is not limited thereto. By using the vacuum-assisted foaming technique, it is possible to form a pore by sufficiently mixing the ceramic slurry added with a polymer serving as a foaming agent and a binder, at the same time, followed by casting.

In the preparation method of the present invention, Step 4 may be performed by subjecting the molded body from which moisture is removed to primary pre-sintering at a temperature of less than 1150° C., specifically, 900° C. to 1100° C. In addition, Step 6 may be performed by subjecting the coated molded body to secondary complete sintering at a temperature of 1100° C. to 1300° C. for 2 hours to 6 hours. At this time, it is presumed that the temperature at the time of performing the primary pre-sintering is lower than the temperature at the time of performing the secondary complete sintering. When the temperature at the time of performing the primary pre-sintering was higher than the temperature at the time of performing the second complete sintering, it was confirmed that the bioactive material aggregated and was unevenly coated. By performing the secondary complete sintering and the primary pre-sintering at a temperature lower than the temperature of the secondary complete sintering, the bioactive material is uniformly coated without aggregation, thereby improving the surface bonding force between the bioactive material and the ceramic surface. In one embodiment of the present invention, it was confirmed by comparison with the Comparative Example that the objects of the present invention could be achieved only if all conditions were satisfied, that is, not only the primary sintering and the secondary sintering must be performed in the preparation method of the present invention, but also, the temperature of the primary sintering must be lower than the temperature of the secondary sintering (FIG. 3).

In the preparation method of the present invention, the bioactive material in Step 5 may include at least one selected from the group consisting of calcium silicate, bioglass, and tricalcium phosphate (TCP), but is not limited thereto. Specifically, the calcium silicate may be β-calcium silicate, but is not limited thereto.

In the preparation method of the present invention, Step 5 may be performed by using a solution containing the bioactive material at a concentration of 1.0 wt % or less, specifically, 0.01 wt % to 0.5 wt %, more specifically, 0.01 wt % to 0.3 wt %. In addition, the coating solution used for coating can be prepared by stirring for 4 hours to 48 hours. More specifically, the uniformity was the highest and the bonding force was also increased, when the stirring time was 48 hours.

In the preparation method of the present invention, the bioactive material may be pulverized by a wet-pulverizing method, but is not limited thereto. At this time, when the wet-pulverizing method is used, a powder having a uniform particle size may be prepared.

In the preparation method of the present invention, the average particle size of the pulverized bioactive material may be 10 μm or less, specifically 0.2 μm to 5 μm, and more specifically, 1 μm to 3 μm. At this time, if the average particle size is 2 μm and the maximum particle size is 10 μm or less, the bioactive material does not sink even if the solvent concentration is lowered and thus no aggregation of the coating solution is observed, and also, the drying time is shortened.

As used herein, the term “glass-ceramic composite” refers to a composite, in which at least one bioactive material selected from the group consisting of calcium silicate, bioglass, and tricalcium phosphate (TCP), is coated on a ceramic surface prepared as a slurry containing at least one ceramic powder selected from the group consisting of hydroxyapatite (HA), biphasic calcium phosphate (BCP), calcium triphosphate, and zirconia, but the glass-ceramic composite is not limited thereto.

As used herein, the term “bioactive material” may refer to a powder which can produce apatite upon sintering at a high temperature and which is composed of components that exhibit bioactivity at the time of bone graft, but the bioactive material is not limited thereto.

As used herein, the term “hydroxyapatite” is a naturally-occurring mineral form of calcium apatite, having the chemical formula of Ca₅(PO₄)₃(OH), but is usually written as Ca₁₀(PO₄)₆(OH)₂ to denote that the crystal unit cell includes two entities. Hydroxyapatite is the hydroxyl endmember of the complex apatite group. The OH-ion can be replaced by fluoride, chloride, or carbonate to produce fluorapatite or chlorapatite. A pure hydroxyapatite powder may appear as white; however, naturally-occurring apatite may appear as brown, yellow, or green. Hydroxyapatite may be naturally produced, but also can be synthesized by wet-chemical deposition, biomimetic deposition, wet-chemical precipitation, also known as sol-gel route, or electrodeposition. Hydroxyapatite can be present in the teeth and bone tissues in the human body, and thus can be used as a filler that replaces a cut bone tissue, or as a coating agent to promote the ingrowth of bone tissues into a prosthetic implant.

In second aspect, the present invention provides a glass-ceramic composite having a structure in which a bioactive material is coated on the surface of the ceramic molded body, wherein the bioactive material has an average particle size of 10 μm or less and is embedded in the surface of a ceramic molded body.

The detailed description of the particles of the bioactive material is as described above. The glass-ceramic composite of the present invention may be prepared by the preparation method including: Step 1 of preparing a slurry containing a ceramic powder;

Step 2 of forming a pore in a molded body by casting the slurry;

Step 3 of removing moisture by freeze-drying the molded body having the pore;

Step 4 of subjecting the molded body from which moisture is removed to primary sintering at 900° C. to 1100° C.;

Step 5 of coating the primary sintered molded body with a bioactive material; and

Step 6 of subjecting the coated molded body to secondary sintering at 1100° C. to 1300° C.

The detailed description of the preparation method is as described above.

In third aspect, the present invention provides a bone grafting material including the glass-ceramic composite having a structure in which a bioactive material is coated on the surface of the ceramic molded body, wherein the bioactive material has an average particle size of 10 μm or less and is embedded in the surface of a ceramic molded body.

As used herein, the “embedded structure” refers to a structure in which a part of the bioactive material particles located at the interface is embedded in the ceramic molded body and the remaining part protrudes outside the surface of the ceramic molded body, and accordingly, the finally-formed composite has a non-flat interface where the layers composed of these two components are not clearly distinguished.

The detailed description of the particles of the bioactive material is as described above.

As used herein, the term “bone grafting material” is also referred to as a ‘bone filler’, a ‘bone substitute material’, or a ‘bone support’, and may also refer to performing a bone graft when a part of bone tissues in the body is defective or needs reinforcement.

In fourth aspect, the present invention provides a bone grafting kit including the glass-ceramic composite having a structure in which a bioactive material is coated on the surface of the ceramic molded body, wherein the bioactive material has an average particle size of 10 μm or less and is embedded in the surface of a ceramic molded body.

The detailed description of the particles of the bioactive material is as described above.

The bone grafting kit of the present invention may further include a means for injecting into the body.

The kit may be used by preparing a paste by mixing the powder with liquid immediately before use, followed by injecting the paste into the desired treatment area.

A syringe may be used as a means for injecting into the body at the time of the treatment.

In addition, the kit of the present invention may further include a biological protein, a drug, or a combination thereof, and the biological protein may include bovine serum albumin, lysozyme, or growth factors, and the drug may include antibiotics, inflammatory agents, etc. without limitations.

The bone grafting material and kit of the present invention can be effectively used as hard tissue substitute materials in orthopedics or dentistry.

### Advantageous Effects

The present invention can provide a bone grafting material having excellent biocompatibility, bone-forming ability, antimicrobial activity, and improved wettability by coating the primary pre-sintered molded body onto the bioactive material, followed by secondary complete sintering such that the bioactive material is uniformly coated, thereby enhancing the surface bonding force between the bioactive material and the ceramic surface at the time of preparing a glass-ceramic composite.

### BRIEF DESCRIPTION OF DRAWINGS

FIG. 1 is a schematic representation of the preparation method of the present invention.

FIG. 2 shows the particle size distribution of calcium silicate powder according to the pulverization time.

FIG. 3 shows the surface of the coating layer of the present invention and the prior art by comparison.

FIG. 4 shows the pH of the glass-ceramic composites prepared using the coating solution at various concentrations according to the elution time.

FIG. 5 shows the surface of the glass-ceramic composites coated with the coating solution at various concentrations before coating.

FIG. 6 shows the SEM images of the surface of the molded body prepared with biphasic calcium phosphate powder before coating and the surface of the glass-ceramic composite coated with bioglass.

FIG. 7 shows the SEM images of the surface of the molded body prepared with biphasic calcium phosphate powder before coating and the surface of the glass-ceramic composite coated with calcium silicate.

FIG. 8 shows the SEM images of the surface of the molded body prepared with hydroxyapatite powder before coating and the surface of the glass-ceramic composite coated with calcium silicate and bioglass.

FIG. 9 shows the SEM images of the surface of the molded body prepared with zirconia powder before coating and the surface of the glass-ceramic composite coated with bioglass.

FIG. 10 shows the SEM images of the surface of the molded body prepared with biphasic calcium phosphate powder before coating and the surface of the glass-ceramic composite coated with calcium silicate.

FIG. 11 shows the specimens implanted with the bone grafting material prepared by coating with the coating solution at various concentrations.

FIG. 12 shows the result of evaluating bone-forming ability after implantation of the bone grafting material prepared by coating with the coating solution at various concentrations.

### DETAILED DESCRIPTION OF EMBODIMENTS

Hereinafter, preferred Examples are provided to help understanding of the present invention. However, these Examples are given for illustrative purposes only to help understanding of the present invention and the scope of the invention is not intended to be limited to or by these Examples.

### PREPARATION EXAMPLE 1

### Preparation of Ceramic Powder

A ceramic powder, in which biphasic calcium phosphate (BCP), hydroxyapatite (HA), β-tricalcium phosphate (β-TCP) and zirconia, and hydroxyapatite and β-tricalcium phosphate were mixed in a ratio of 6:4, was prepared.

### PREPARATION EXAMPLE 2

### Preparation of Coating Solution

Calcium silicate was prepared as a bioactive material. The thus-prepared calcium silicate was subjected to wet-pulverization to have an average particle size of 2 μm and a maximum particle size of 10 μm or less (FIG. 2). Polyvinyl butyral (PVB) was added as a solvent, and the mixture was stirred for 8 hours to 48 hours to obtain calcium silicate solution at concentrations of 0.1 wt %, 0.2 wt %, 0.3 wt %, 0.4 wt %, 0.5 wt %, and 1.0 wt %.

When the average particle size was 2 μm and the maximum particle size was 10 μm or less, the bioactive material did not sink even if the solvent concentration was lowered and thus no aggregation of the coating solution was observed, and also, the drying time was shortened. Further, it was confirmed that the uniformity was the highest, when the stirring time was 48 hours.

Bioglass and β-tricalcium phosphate were prepared as bioactive materials.

### Example 1

### Preparation of Glass-Ceramic Composites

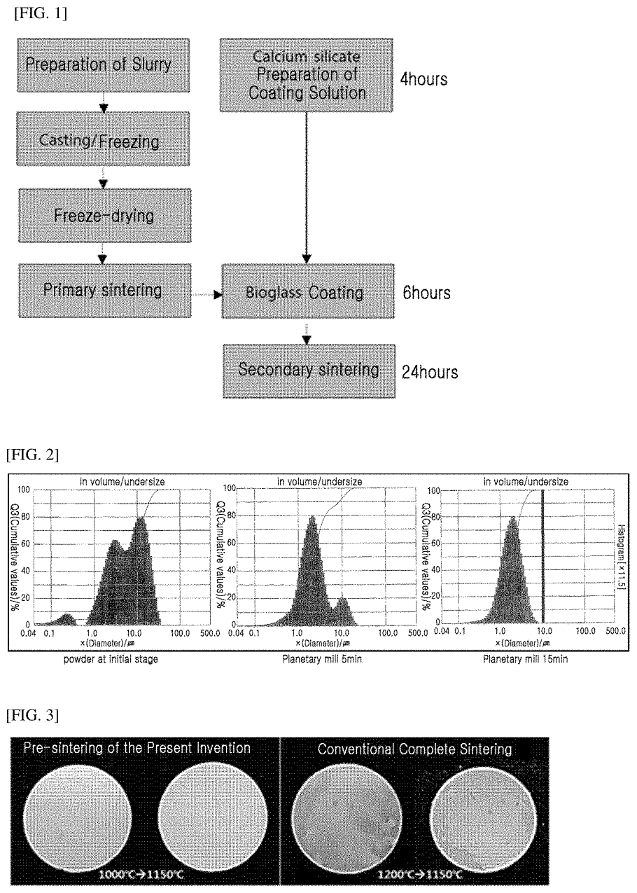

A slurry was prepared using the ceramic powder, in which hydroxyapatite and β-trisodium phosphate were mixed at a ratio of 6:4. The thus-prepared slurry was casted to form pores in the molded body, and then freeze-dried to remove moisture. After subjecting the molded body from which moisture was removed to primary pre-sintering at 1,000° C., and the pre-sintered molded body was coated with the calcium silicate solution at concentrations of 0.1 wt %, 0.2 wt %, 0.3 wt %, 0.4 wt %, 0.5 wt % and 1.0 wt % prepared in Preparation Example 2. The coated molded body was dried and then subjected to secondary sintering at 1,150° C. for 4 hours to prepare a glass-ceramic composite (a schematic representation of FIG. 1). The SEM image of the surface of the prepared glass-ceramic composite is shown in FIG. 5.

Glass-ceramic composites were prepared in the same manner as described above, except that the slurry was prepared using biphasic calcium phosphate, hydroxyapatite, β-tricalcium phosphate, or zirconia as a ceramic powder.

Glass-ceramic composites were prepared as described above except that the molded body was coated with bioglass, β-tricalcium phosphate or bioglass, and silicate instead of calcium silicate.

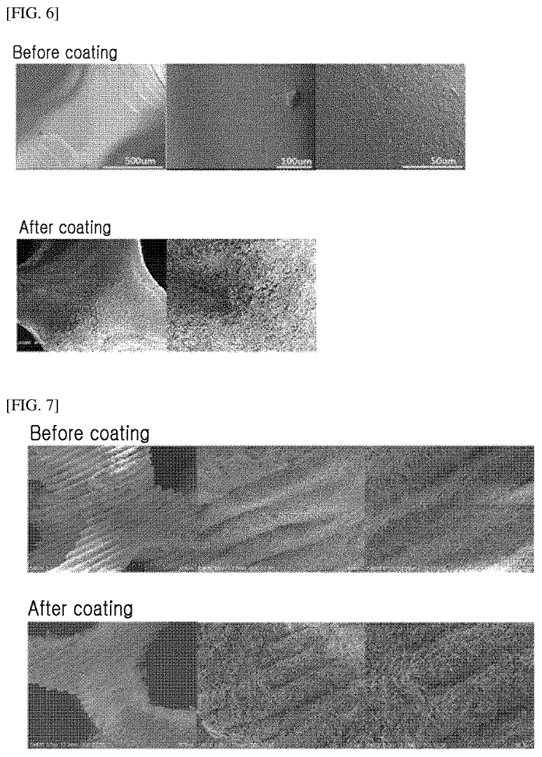

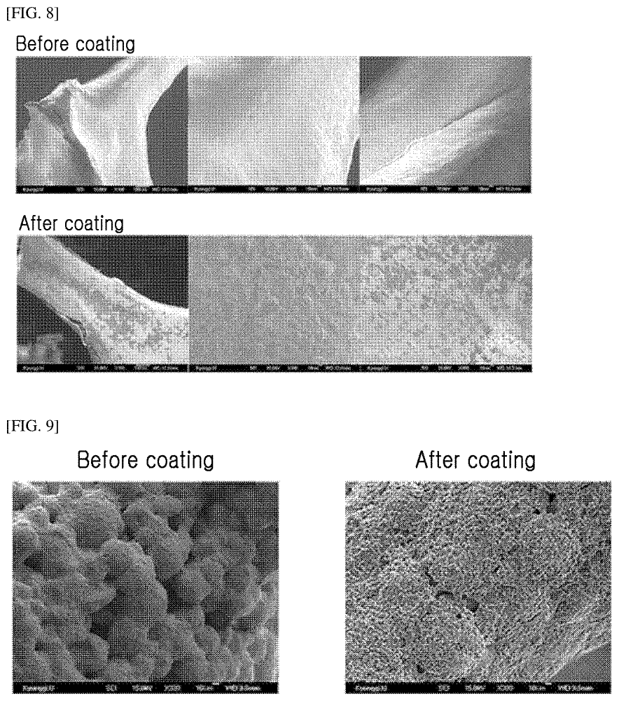

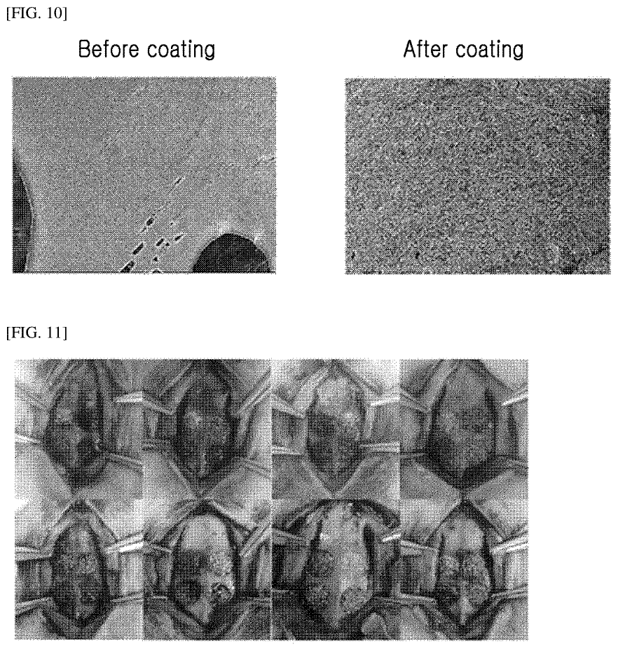

Among the glass-ceramic composites prepared above, representatively, the SEM images of the surfaces of the glass-ceramic composite prepared by coating the molded body prepared using biphasic calcium phosphate powder with bioglass, the glass-ceramic composite prepared by coating the molded body prepared using biphasic calcium phosphate with calcium silicate, the glass-ceramic composite prepared by coating the molded body prepared using hydroxyapatite powder with calcium silicate and bioglass, the glass-ceramic composite prepared by coating the molded body prepared using zirconia powder with bioglass, and the glass-ceramic composite prepared by coating the molded body prepared using biphasic calcium phosphate powder with calcium silicate are shown in FIGS. 6 to 10, respectively.

### Comparative Example 1

### Preparation of Glass-Ceramic Composite in which the Temperature of Pre-sintering is Higher Than the Temperature of Complete Sintering

A glass-ceramic composite, in which the molded body prepared using a ceramic powder prepared by mixing hydroxyapatite and β-tricalcium phosphate at a ratio of 6:4 was coated with calcium silicate, was prepared in the same manner as in Example 1, except the molded body from which moisture is removed was subjected to primary pre-sintering at 1,200° C. instead of 1,000° C.

As shown in FIG. 3, in the case of the glass-ceramic composite prepared in Example 1, in which the molded body prepared using a ceramic powder prepared by mixing hydroxyapatite and β-tricalcium phosphate at a ratio of 6:4 was coated with calcium silicate, it was confirmed that the surface of the glass-ceramic composite was uniformly coated without aggregation as compared with the glass ceramic composite of Comparative Example 1 prepared by performing pre-sintering at a temperature higher that the temperature of complete sintering. Further, it was confirmed that the coating was most uniform at a low concentration (concentration of 0.2 wt % or less), and the bonding force was also high (FIG. 5).

### Experimental Example 1

### pH Analysis

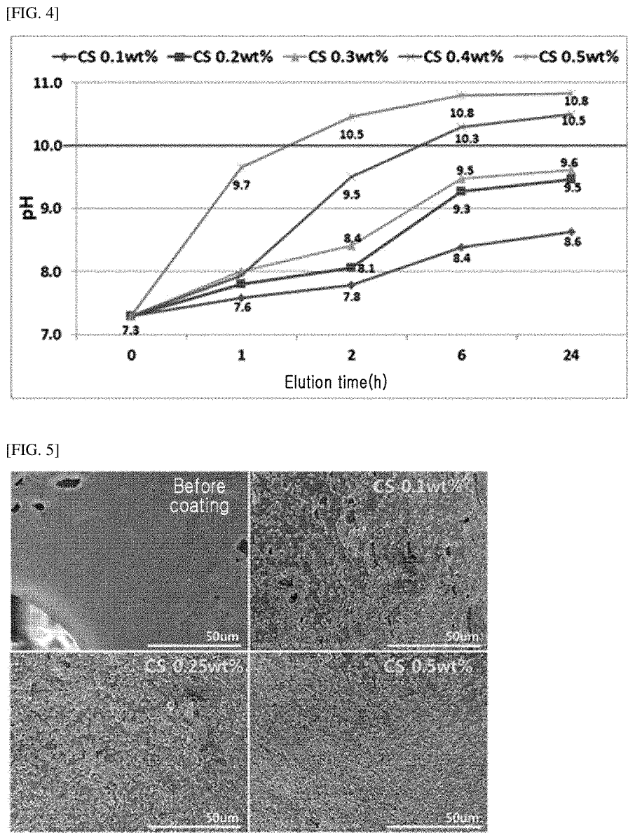

In order to analyze the excessive increase of pH due to the material eluted from the coated glass-ceramic composites as time elapsed, the pH of the glass-ceramic composites coated with the calcium silicate solution at concentrations of 0.1 wt %, 0.2 wt %, 0.3 wt %, 0.4 wt %, 0.5 wt % and 1.0 wt % was analyzed. When the glass-ceramic composites were coated with the calcium silicate solution at a concentration of 0.3 wt % or less, the pH was 10 or less, while when the composites were coated with the calcium silicate solution at concentrations of 0.4 wt % and 0.5 wt %, the pH excessively rose to 10 or more (FIG. 4). In particular, it was confirmed that the pH excessively rose to 11 when the glass-ceramic composite was coated with the calcium silicate solution at a concentration of 1.0 wt %.

### Experimental Example 2

### Confirmation of Inflammation Reaction

In rabbit calvarial defect models, inflammation reaction of the bone grafting material was evaluated by 4- and 8-week animal studies. As a result of implanting the bone grafting material prepared using the glass-ceramic composites of Example 1 coated with the calcium silicate solution at concentrations of 0.1 wt %, 0.2 wt %, 0.3 wt %, 0.4 wt %, 0.5 wt % and 1.0 wt %, it was confirmed that no inflammation reaction occurred in all specimens (FIG. 11).

### Experimental Example 3

### Analysis of Osteogenic Capacity

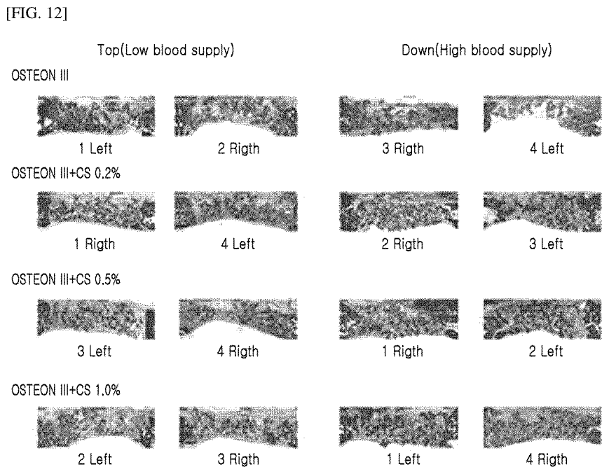

In rabbit calvarial defect models, the analysis of osteogenic capacity was evaluated by 4- and 8-week animal studies. As a result of implanting the bone grafting material prepared using the ceramic powder (OSTEON 3) prepared by mixing hydroxyapatite and β-tricalcium phosphate at a ratio of 6:4 and the calcium silicate solution at concentrations of 0.2 wt %, 0.5 wt % and 1.0 wt %, it was confirmed that bone formation was excellent in all specimens (FIG. 12). In particular, it was confirmed that the osteogenic capacity was the highest in case of Example 1 coated with 0.2 wt % of calcium silicate solution.

### ACKNOWLEDGEMENT

This work has been supported by the Innopolis Foundation grant funded by the Korea government(MSIT)(No. 2017-DD-RD-0030-02).

1. A method for preparing a glass-ceramic composite, comprising:
Step 1 of preparing a slurry containing a ceramic powder;
Step 2 of forming a pore in a molded body by casting the slurry;
Step 3 of removing moisture by freeze-drying the molded body having the pore;
Step 4 of subjecting the molded body from which moisture is removed to primary sintering at 900° C. to 1100° C.;
Step 5 of coating the primary sintered molded body with a bioactive material; and
Step 6 of subjecting the coated molded body to secondary sintering at 1100° C. to 1300° C.

2. The method of claim 1, wherein the bioactive material in Step 5 comprises at least one selected from the group consisting of calcium silicate, bioglass, and tricalcium phosphate (TCP).

3. The method of claim 2, wherein the bioactive material is pulverized by a wet-pulverizing method.

4. The method of claim 3, wherein the pulverized bioactive material has an average particle size of 10 μm or less.

5. The method of claim 2, wherein Step 5 is performed by using a solution containing the bioactive material at a concentration of 0.01 wt % to 0.5 wt %.

6. The method of claim 1, wherein the ceramic powder in Step 1 comprises at least one selected from the group consisting of hydroxyapatite (HA), biphasic calcium phosphate (BCP), tricalcium phosphate, and zirconia.

7. A glass-ceramic composite having a structure in which a bioactive material is coated on the surface of the ceramic molded body, wherein the bioactive material has an average particle size of 10 μm or less and is embedded in the surface of a ceramic molded body.

8. The glass-ceramic composite of claim 7, wherein the glass-ceramic composite is prepared by the preparation method of any one of claims 1 to 6.

9. A bone grafting material comprising the glass-ceramic composite of claim 7.

10. A bone grafting kit comprising the glass-ceramic composite of claim 7.

11. The kit of claim 10, further comprising a means for injecting into the body.
